(19) United States
(12) Patent Application Publication
(10) Pub. No.: US 2026/0327166 A1
(43) Pub. Date: Oct. 22, 2026

(54) INTEGRATED PROCESS FOR MAKING E-1-CHLORO-3,3,3-TRIFLUOROPROPENE (HCFO-1233zd(E)) FROM A MIXTURE OF HIGH-BOILING FLUORINATED COMPONENTS

(71) Applicant: Solstice Advanced Materials US, Inc., Morris Plains, NJ (US)

(72) Inventors: Daniel C. Merkel, Orchard Park, NY (US); Haiyou Wang, Amherst, NY (US); Jennifer W. McClaine, Branchburg, NJ (US)

(21) Appl. No.: 19/648,448

(22) Filed: Apr. 15, 2026

Related U.S. Application Data
(60) Provisional application No. 63/790,195, filed on Apr. 17, 2025.

(51) Int. Cl. C07C 17/087; C07C 17/383

(52) U.S. Cl. CPC C07C 17/087; C07C 17/383

(57) ABSTRACT

A method for conversion of a mixture of high-boiling fluorinated components to E-1-chloro-3,3,3-trifluoropropene (HCFO-1233zd(E)) is described. The mixture of high-boiling fluorinated components may include 1,1,3,3-tetrachloro-1-fluoropropane (HCFC-241fa), 1,3,3-trichloro-1,1-difluoropropane (HCFC-242fa), 1,1,3-trichloro-1,3-difluoropropane (HCFC-242fb), 3,3-dichloro-1,1,1-trifluoropropane (HCFC-243fa), 1,3-dichloro-1,1,3-trifluoropropane (HCFC-243fb), 3-chloro-1,1,1,3-tetrafluoropropane (HCFC-244fa), and their isomers. Heavy impurities, such as oligomers and other high-boiling undesired byproducts, such as hexachloroethane, that are present may be separated from the recyclable intermediates through use of a middle boiler column and purged during the process to prevent yield loss and reduction of catalyst efficacy.

### CROSS-REFERENCE TO RELATED APPLICATION

This application claims priority to U.S. Provisional Application No. 63/790,195 filed Apr. 17, 2025, which is herein incorporated by reference in its entirety.

### FIELD

The present disclosure is related to an integrated process for making E-1-chloro-3,3,3-trifluoropropene (HCFO-1233zd(E)) from a mixture of high-boiling fluorinated components.

### BACKGROUND

Chlorofluorocarbons (CFCs) like trichlorofluoromethane and dichlorodifluoromethane have been used as refrigerants, blowing agents and diluents for gaseous sterilization. Many years ago it was determined that chlorofluorocarbons are detrimental to the Earth's ozone layer and have high Global Warming Potential. As a result, there is a worldwide effort to use halocarbons which contain fewer or no chlorine substituents. Accordingly, the production of hydrofluorocarbons, or compounds containing only carbon, hydrogen, and fluorine, has been the subject of increasing interest to provide environmentally desirable products for use as solvents, blowing agents, refrigerants, cleaning agents, aerosol propellants, heat transfer media, dielectrics, fire extinguishing compositions and power cycle working fluids.

In this regard, E-1-chloro-3,3,3-trifluoropropene (HCFO-1233zd(E)) is a compound that has the potential to be used as a zero Ozone Depletion Potential (ODP) and a low Global Warming Potential (GWP) refrigerant, blowing agent, aerosol propellant, solvent, etc., and as a fluorinated monomer.

### SUMMARY

The present disclosure provides a method for conversion of a mixture of high-boiling fluorinated components to E-1-chloro-3,3,3-trifluoropropene (HCFO-1233zd(E)). The mixture of high-boiling fluorinated components may comprise 1,1,3,3-tetrachloro-1-fluoropropane (HCFC-241fa), 1,3,3-trichloro-1,1-difluoropropane (HCFC-242fa), 1,1,3-trichloro-1,3-difluoropropane (HCFC-242fb), 3,3-dichloro-1,1,1-trifluoropropane (HCFC-243fa), 1,3-dichloro-1,1,3-trifluoropropane (HCFC-243fb), 3-chloro-1,1,1,3-tetrafluoropropane (HCFC-244fa), their isomers, and combinations thereof. Heavy impurities, such as oligomers and other high-boiling undesired byproducts, such as hexachloroethane, that are present may be separated from the recyclable intermediates through use of a middle boiler column and purged during the process to prevent yield loss and reduction of catalyst efficacy. The recyclable intermediates are recycled back to the feed tank and mixed with the high-boiling fluorinated components in the feed stock.

In one form thereof, a method for producing E-1-chloro-3,3,3-trifluoropropene (HCFO-1233zd(E)) comprises: providing a mixture of high-boiling fluorinated components having a normal boiling point of greater than 39 °C; contacting the mixture of high-boiling fluorinated components with hydrogen fluoride (HF) to produce a combined reactant stream; reacting the combined reactant stream in the presence of a catalyst to produce a product stream comprising E-1-chloro-3,3,3-trifluoropropene (HCFO-1233zd(E)); recovering, from the product stream, a high-boiling intermediate stream comprising recyclable high-boiling intermediates having a normal boiling point of greater than 39 °C and less than 140 °C; and recycling the recyclable high-boiling intermediates into the mixture of high-boiling fluorinated components prior to the reacting step.

### BRIEF DESCRIPTION OF THE DRAWING

The above mentioned and other features of the disclosure, and the manner of attaining them, will become more apparent and the disclosure itself will be better understood by reference to the following description of embodiments of the disclosure taken in conjunction with the accompanying drawing.

FIG. 1 shows a schematic of a process to synthesize E-1-chloro-3,3,3-trifluoropropene (HCFO-1233zd(E)) from a mixture of high-boiling fluorinated components.

Corresponding reference characters indicate corresponding parts throughout the several views. Although the drawing represents embodiments of various features and components according to the present disclosure, the drawing is not necessarily to scale and certain features may be exaggerated to better illustrate and explain the present disclosure. The exemplification set out herein illustrates an embodiment of the disclosure, and such exemplification is not to be construed as limiting the scope of the disclosure in any manner.

### DETAILED DESCRIPTION

### Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

As used herein, the phrase “high-boiling fluorinated components” refer to fluorinated components with normal boiling points higher than 39 °C; in other words, normal boiling points higher than that of Z-1-chloro-3,3,3-trifluoropropene (HCFO-1233zd(Z)). The high-boiling fluorinated components may be present in the feed stream before entering the reactor and/or the product stream from the reactor and may include desired intermediates and/or undesired byproducts.

As used herein, the phrase “desired product” refers to E-1-chloro-3,3,3-trifluoropropene (HCFO-1233zd(E)).

As used herein, the phrase “low-boiling impurities” refer to impurities having normal boiling points lower than 39 °C, such as 1,1,1,3,3-pentafluoropropane (HFC-245fa), trifluoropropyne, and E- and Z-isomers of 1,3,3,3-tetrafluoropropene (HFO-1234ze), among others.

As used herein, the phrase “desired high-boiling intermediates” refer to components that have a normal boiling point higher than 39 °C but lower than 140 °C, such as 1,1,3,3-tetrachloro-1-fluoropropane (HCFC-241fa), 1,3,3-trichloro-1,1-difluoropropane (HCFC-242fa), 1,1,3-trichloro-1,3-difluoropropane (HCFC-242fb), 3,3-dichloro-1,1,1-trifluoropropane (HCFC-243fa), 1,3-dichloro-1,1,3-trifluoropropane (HCFC-243fb), C₃Cl₃F₂H (HCFO-1232 isomers, for example, 1,3-dichloro-3,3-difluoropropene (HCFO-1232zd)), and C₃Cl₄FH (HCFO-1231 isomers, for example, 1,3,3-trichloro-3-fluoropropene (HCFO-1231zd)). Desired high-boiling intermediates can be recycled and/or further reacted to produce the desired product using the methods described herein.

As used herein, the phrase “undesired high-boiling byproducts” refer to components that have a normal boiling point higher than 140 °C, such as oligomers and other high-boiling undesired byproducts, such as hexachloroethane. Undesired high-boiling byproducts cannot be easily further reacted to produce the desired product using the methods described herein.

As used herein, conversion of a reactant molecule (molecule X) during a reaction is calculated using the following equation when substantially pure reactant is used:

% conversion of molecule X=(100−molecule X mol. % in the organic components of a product mixture)

When the reactant includes impurities or recycled components, i.e., is a component in a reaction mixture, the conversion of a reactant molecule (molecule X) during a reaction is calculated using the following equations:

% conversion of moleculeX=(change in X mol.%)/(Xmol % in the reactant mixture)

OR

% conversion of molecule X=(X mol % in the reactant mixture −X mol % in the product mixture)/(X mol % in the reactant mixture)

As used herein, selectivity to a molecule formed during a reaction (molecule X) is calculated using the following equation:

% selectivity to molecule X=mol.% of molecule X in the organic components of a product mixture/(100-mol.% of reactant molecules in the organic components of a product mixture)×100.

### Overview

The present disclosure provides a process for the production of E-1-chloro-3,3,3-trifluoropropene (HCFO-1233zd(E)) from a mixture of high-boiling fluorinated components. Specifically, the present disclosure provides a method for manufacturing E-1-chloro-3,3,3-trifluoropropene (HCFO-1233zd(E)) from high-boiling fluorinated components including one or more of isomers of HCFC-241, HCFC-242, HCFC-243, and HCFC-244 which are produced as intermediates from a process in which 1-chloro-3,3,3-trifluoropropene (HCFO-1233zd) is produced from 1,1,1,3,3-pentachloropropene (HCC-240fa). By the present method, the combined stream of intermediates may then be converted to E-1-chloro-3,3,3-trifluoropropene (HCFO-1233zd(E)).

1-chloro-3,3,3-trifluoropropene (HCFO-1233zd) may be produced industrially by the reaction shown below in Equation 1.

HCC-240fa+3HF→HCFO-1233zd+4HCl Equation 1:

In this process, 1-chloro-3,3,3-trifluoropropene (HCFO-1233zd) may be produced in a liquid phase reaction without the need for a catalyst. Briefly, anhydrous hydrogen fluoride (HF) may be mixed with 1,1,1,3,3-pentachloropropene (HCC-240fa) at elevated temperature and pressure to produce the desired 1-chloro-3,3,3-trifluoropropene (HCFO-1233zd) in a mixture of E- and Z-isomers.

In the course of the reaction, partially fluorinated components such as 1,1,3,3-tetrachloro-1-fluoropropane (HCFC-241fa), 1,3,3-trichloro-1,1-difluoropropane (HCFC-242fa), 3,3-dichloro-1,1,1-trifluoropropane (HCFC-243fa), and 3-chloro-1,1,1,3-tetrafluoropropane (HCFC-244fa), along with their other isomers, may be produced according to Equations 2-5, shown below.

HCC-240fa+HF→HCFC-241 isomers+HCl Equation 2:

HCC-240fa+2HF→HCFC-242 isomers+2HCl Equation 3:

HCC-240fa+3HF→HCFC-243 isomers+3HCl Equation 4:

HCC-240fa+4HF→HCFC-244 isomers+4HCl Equation 5:

The mixture of high-boiling fluorinated components may be defined as a mixture of fluorinated components with normal boiling points higher than 39 °C; in other words, normal boiling points higher than that of Z-1-chloro-3,3,3-trifluoropropene (HCFO-1233zd(Z)).

The high-boiling fluorinated components may include desired high-boiling intermediates that may be converted to 1-chloro-3,3,3-trifluoropropene (HCFO-1233zd) using the methods described herein. Suitable such desired high-boiling intermediates may include 1,1,3,3-tetrachloro-1-fluoropropane (HCFC-241fa), 1,3,3-trichloro-1,1-difluoropropane (HCFC-242fa), 3,3-dichloro-1,1,1-trifluoropropane (HCFC-243fa), and 3-chloro-1,1,1,3-tetrafluoropropane (HCFC-244fa), along with their other isomers. For example, another isomer of HCFC-242 is 1,1,3-trichloro-1,3-difluoropropane (HCFC-242fb), and another isomer of HCFC-243 is 1,3-dichloro-1,1,3-trifluoropropane (HCFC-243fb).

The desired high-boiling intermediates may be converted to 1-chloro-3,3,3-trifluoropropene (HCFO-1233zd) according to Equations 6-14, shown below.

CF₃CH₂CHFCl (244fa)→CF₃CH═CHCl (1233zd)+HF Equation 6:

Cis-CF₃CH═CHCl (1233zdZ)→Trans-CF₃CH═CHCl (1233zdE) Equation 7:

C₃Cl₃F₂H (1232 isomers)+HF→CF₃CH═CHCl (1233zd)+HCl Equation 8:

CF₃CH₂CHCl₂(243fa)→CF₃CH═CHCl (1233zd)+HCl Equation 9:

CF₂ClCH₂CHFCl (243fb)+HF→CF₃CH═CHCl (1233zd)+HCl+HF Equation 10:

CF₂ClCH₂CHCl₂(242fa)+HF+CF₃CH═CHCl (1233zd)+2HCl Equation 11:

CFCl₂CH₂CHFCl (242fb)+2HF→CF₃CH═CHCl (1233zd)+2HCl+HF Equation 12:

C₃Cl₄FH (1231 isomers)+2HF→CF₃CH═CHCl (1233zd)+HCl Equation 13:

CFCl₂CH₂CHCl₂(241fa)+2HF→CF₃CH═CHCl (1233zd)+3HCl Equation 14:

Undesired high-boiling byproducts that may be present in the mixture of high-boiling fluorinated components include oligomers. Oligomers, as used herein, may be defined as dimers, trimers, tetramers, and other groups of repeated and/or mixed monomeric units that may not be easily converted to 1-chloro-3,3,3-trifluoropropene (HCFO-1233zd) using the methods described herein.

Other impurities that may also be present in the reactor as undesired high-boiling byproducts include very high boiling impurities; in other words, impurities with normal boiling points of greater than about 140 °C. These very high boiling impurities may include CC-110 (hexachloroethane), HCO-1230 (tetrachloropropene) isomers, and some oligomers. One example of HCO-1230 isomers may be HCO-1230xa (1,1,2,3-tetrachloropropene). Possible examples of oligomers may be dimers of 1,3,3-trichloro-3-fluoropropene (HCFO-1231zd) and 1,3-dichloro-3,3-difluoropropene (HCFO-1232zd), with the chemical formula C₆H₃F₃Cl₄. For the purpose of this invention, these very high boiling impurities or high-boiling byproducts, including CC-110, HCO-1230, and oligomers, may also be referred to as heavy impurities.

The present disclosure provides a method of maximizing the production efficiency of E-1-chloro-3,3,3-trifluoropropene (HCFO-1233zd(E)) by reacting a mixture of high-boiling fluorinated components with HF in the presence of a catalyst to produce 1-chloro-3,3,3-trifluoropropene (HCFO-1233zd), and removing undesired high-boiling byproducts.

### Process

Briefly, the reactor products from Equation 1 are first separated to recover and recycle excess HF and to remove and recover the hydrogen chloride (HCl). The remaining mixture comprises E- and Z-isomers of 1-chloro-3,3,3-trifluoropropene (HCFO-1233zd); high-boiling fluorinated components; low boiling impurities such as 1,1,1,3,3-pentafluoropropane (HFC-245fa), trifluoropropyne, and E- and Z-isomers of 1,3,3,3-tetrafluoropropene (HFO-1234ze), among others; and a small amount of residual HF (usually 2 wt. % or less). This mixture may be subjected to distillation to remove the high-boiling fluorinated components from the 1-chloro-3,3,3-trifluoropropene (HCFO-1233zd), low boiling impurities, and HF.

Following distillation, the vapor phase high-boiling fluorinated components may be passed through another reactor together with HF. The reactor may include a catalyst. Without wishing to be bound by theory, it is possible that the presence of heavy impurities in the reactor may reduce catalyst activity. Therefore, prior to entering the reactor, undesired heavy impurities may be purged. Following the reaction, any heavy impurities created may be purged prior to purification of the desired product.

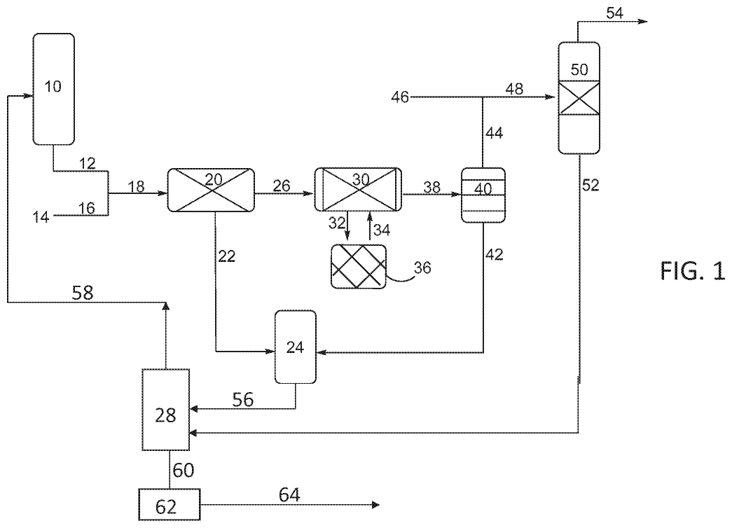

An example of the process is shown in FIG. 1 and summarized below. As shown therein, a stream 12 of the high-boiling fluorinated components (e.g., isomers of HCFC-241, HCFC-242, HCFC-243, HCFC-244) and heavy impurities recovered from the reaction of 1,1,1,3,3-pentachloropropene (HCC-240fa) and anhydrous HF (not shown) stored in high boiler feed tank 10 may be combined with a stream 16 comprising HF from an HF feed supply source 14 (e.g., an HF feed tank). The combined stream 18 may be passed to a vaporizer/superheater 20.

The resulting superheated gaseous stream 26 comprising the vaporized high-boiling fluorinated components and HF are fed to vapor phase reactor 30. Unvaporized high-boiling fluorinated components in stream 22 comprising mainly high molecular weight organics (oligomers), are separated from vaporizer/superheater 20 and conveyed to a high molecular weight (oligomer) knock out tank 24 to prevent them from entering the reactor 30.

The high-boiling fluorinated components from stream 22 in the knock out tank 24 may be treated in the middle boiler column 28, for example by distillation such as distillation under vacuum, to recover desired high-boiling intermediates such as 1,1,3,3-tetrachloro-1-fluoropropane (HCFC-241fa), 1,3,3-trichloro-1,1-difluoropropane (HCFC-242fa), 1,1,3-trichloro-1,3-difluoropropane (HCFC-242fb), 3,3-dichloro-1,1,1-trifluoropropane (HCFC-243fa), 1,3-dichloro-1,1,3-trifluoropropane (HCFC-243fb), and 3-chloro-1,1,1,3-tetrafluoropropane (HCFC-244fa), and their isomers, for example, which can be sent to the reactor 30 for conversion to 1-chloro-3,3,3-trifluoropropene (HCFO-1233zd) product, as well as other useful components such as 1,1,3,3-tetrachloropropene (HCO-1230za) and its isomers, which can be sent to a separate 240fa reactor (not shown) for conversion to 1-chloro-3,3,3-trifluoropropene (HCFO-1233zd) product.

The low-boiling impurities produced during the process may comprise E-1,3,3,3-tetrafluoropropene (HFO-1234ze(E)), Z-1,3,3,3-tetrafluoropropene (HFO-1234ze(Z)), and 1,1,1,3,3-pentafluoropropane (HFC-245fa). Other products produced during the process may comprise 2-chloro-3,3,3-trifluoropropene (HCFO-1233xf); isomers of HCFO-1232, HCFO-1231, and HFO-1230; 1-chloro-1,3,3,3-tetrafluoropropane (HCFC-244fa), trichloropropene isomers, hexachloroethane, dimers such as C₆H₄F₈ isomers, C₆H₃F₇ isomers, C₆H₄F₇Cl isomers, C₆H₃F₆Cl isomers, and C₆H₃F₃Cl₄ isomers, among others.

A heating system 36 may be used to cool 32 or heat 34 the reactor 30. The heating system 36 may comprise any suitable heating medium capable of achieving and/or maintaining the temperatures required by the process. Suitable heating media may include molten salt, hot oil, high pressure steam, and electric heaters (direct, resistance, or induction), among others, for example.

Reactor 30 is a gas phase reactor in which the contents of stream 26 are reacted in the gas phase, per the conditions set forth below. From reactor 30, a crude product stream 38 comprising E- and Z-isomers of 1-chloro-3,3,3-trifluoropropene (HCFO-1233zd), HF, HCl, and the newly formed high-boiling fluorinated components may be passed to a partial condenser 40 to remove high-boiling byproducts from the product stream 38 to produce a first processed stream 44.

The partial condenser 40 may be operated at a temperature of as low as about 10 °C, about 20 °C, about 30 °C, about 40 °C, or as high as about 50 °C, about 60 °C, about 70 °C, about 80 °C, or any value or range encompassed by these endpoints, for example, from about 10 °C to about 80 °C, or from about 20 °C to about 70 °C.

The partial condenser 40 may be operated at a pressure of as little as about 1 bar, about 2 bar, or as great as about 3 bar, about 4 bar, about 5 bar, or any value or range encompassed by these endpoints, for example, from about 1 bar to about 5 bar, or from about 2 bar to about 4 bar. Specific examples of additional suitable ranges for temperature and pressure of the partial condenser 40 are set forth below in Table 1. The values provided in Table 1 are understood to be prefaced by “about”.

TABLE 1
Temperature and Pressure in Partial Condenser 40

| Temperature (°C) | Pressure (bar) |
|---|---|
| 10-80 | 1-5 |
| 10-80 | 1-4 |
| 10-80 | 1-3 |
| 10-80 | 2-4 |
| 10-80 | 2-3 |
| 10-70 | 1-5 |
| 10-70 | 1-4 |
| 10-70 | 1-3 |
| 10-70 | 2-4 |
| 10-70 | 2-3 |
| 20-70 | 1-5 |
| 20-70 | 1-4 |
| 20-70 | 1-3 |
| 20-70 | 2-4 |
| 20-70 | 2-3 |
| 30-80 | 1-5 |
| 30-80 | 1-4 |
| 30-80 | 1-3 |
| 30-80 | 2-4 |
| 30-80 | 2-3 |

The condensed portion from condenser 40, stream 42 comprising high-boiling byproducts, is drained to the high molecular weight (oligomer) knock out tank 24. A gaseous product stream 44 (e.g., a first processed stream) comprising E-isomers of 1-chloro-3,3,3-trifluoropropene (HCFO-1233zd(E)) is combined with a 1233zd(E) crude stream 46 (e.g., from a 1233zd(E) crude feed tank via a separate 1233zd(E) production process, for example, from a reaction of 1,1,1,3,3-pentachloropropene (HCC-240fa) and HF (not shown)) and this combination creates stream 48 which is fed to 1233zd(E) crude distillation column 50 to produce a second product stream comprising HCFO-1233zd(E) and a second processed stream.

The distillation column 50 may be operated at a top distillate temperature of as low as about 10 °C, about 20 °C, about 30 °C, or as high as about 40 °C, about 50 °C, about 60 °C, or any value or range encompassed by these endpoints, for example, from about 10 °C to about 60 °C, or from about 20 °C to about 50 °C.

The distillation column 50 may be operated at a pressure of as little as about 1 bar, about 2 bar, or as great as about 3 bar, about 4 bar, about 5 bar, or any value or range encompassed by these endpoints, for example, from about 1 bar to about 5 bar, or from about 2 bar to about 4 bar. Specific examples of additional suitable ranges for temperature and pressure of the distillation column 50 are set forth below in Table 2. The values provided in Table 2 are understood to be prefaced by “about”.

TABLE 2
Temperature and Pressure in Distillation Column 50

| Temperature (°C) | Pressure (bar) |
|---|---|
| 10-60 | 1-5 |
| 10-60 | 1-4 |
| 10-60 | 2-5 |
| 10-60 | 2-4 |
| 10-50 | 1-5 |
| 10-50 | 1-4 |
| 10-50 | 2-5 |
| 10-50 | 2-4 |
| 20-60 | 1-5 |
| 20-60 | 1-4 |
| 20-60 | 2-5 |
| 20-60 | 2-4 |
| 20-50 | 1-5 |
| 20-50 | 1-4 |
| 20-50 | 2-5 |
| 20-50 | 2-4 |
| 30-50 | 1-5 |
| 30-50 | 1-4 |
| 30-50 | 2-5 |
| 30-50 | 2-4 |

The overhead stream 54 from distillation column 50 is taken off the top of column 50 and comprises mainly HCFO-1233zd(E). The overhead stream 54 may be sent to purification columns (not shown in FIG. 1) to isolate and recover purified E-1-chloro-3,3,3-trifluoropropene (HCFO-1233zd(E)) product and recyclable intermediates comprising Z-1-chloro-3,3,3-trifluoropropene (HCFO-1233zd(Z)) and 3-chloro-1,1,1,3-tetrafluoropropane (HCFC-244fa) which can be further converted to E-1-chloro-3,3,3-trifluoropropene (HCFO-1233zd(E)).

The bottom stream 52 (e.g., a third processed stream) from distillation column 50 comprises 241fa, 242fa, 242fb, 243fa, 243fb, 1232 and 1231 isomers, and is drained from the reboiler and fed to middle boiler column 28. The exit stream 56 from the high molecular weight (oligomer) knock out tank 24 is also drained to middle boiler column 28.

Stream 58 (e.g., a high-boiling intermediate stream) is taken off the top of the middle boiler column 28 and comprises mainly desired high-boiling intermediates 241fa, 242fa, 242fb, 243fa, 243fb, 1232 and 1231 isomers and is fed to high boiler feed tank 10. High-boiling intermediate stream 58 may comprise HCFC-242fa in an amount of at least about 10 wt. %, about 20 wt. %, about 30 wt. %, about 40 wt. %, about 50 wt. %, or as much as about 60 wt. %, about 70 wt. %, about 80 wt. %, about 90 wt. % or within any range encompassed by any two of those values as endpoints, for example from about 10 wt. % to about 90 wt. %, preferably from about 30 wt. % to about 80 wt. %, more preferably from about 50 wt. % to about 70 wt. %. High-boiling intermediate stream 58 may comprise HCFC-243fa in an amount of at least about 5 wt. %, about 10 wt. %, about 15 wt. %, about 20 wt. %, or as great as about 25 wt. %, about 30 wt. %, about 35 wt. %, about 40 wt. %, about 50 wt. %, or within any range encompassed by any two of those values as endpoints, for example from about 5 wt. % to about 50 wt. %, preferably from about 10 wt. % to about 40 wt. %, more preferably from about 15 wt. % to about 30 wt. %. High-boiling intermediate stream 58 may comprise 243fb in an amount of at least about 0.5 wt. %, about 1 wt. %, about 3 wt. %, about 4 wt. %, or as great as about 5 wt. %, about 6 wt. %, about 7 wt. %, about 8 wt. %, about 10 wt. %, about 15 wt. %, or within any range encompassed by any two of those values as endpoints, for example from about 0.5 wt. % to about 15 wt. %, preferably from about 1 wt. % to about 10 wt. %, more preferably from about 3 wt. % to about 7 wt. %. High-boiling intermediate stream 58 may comprise HCFC-1233 in an amount of at least about 0.1 wt. %, about 0.5 wt. %, about 1 wt. %, about 2 wt. %, or as great as about 2.5 wt. %, about 3 wt. %, about 4 wt. %, about 5 wt. %, about 10 wt. %, or within any range encompassed by any two of those values as endpoints, for example from about 0.1 wt. % to about 10 wt. %, preferably from about 1 wt. % to about 3 wt. %. High-boiling intermediate stream 58 may comprise HCFC-1232 (2) in an amount of at least about 0.1 wt. %, about 0.5 wt. %, about 1 wt. %, about 2 wt. %, or as great as about 2.5 wt. %, about 3 wt. %, about 4 wt. %, about 5 wt. %, about 10 wt. %, or within any range encompassed by any two of those values as endpoints, for example from about 0.1 wt. % to about 10 wt. %, preferably from about 0.5 wt. % to about 3 wt. %. All weight percentages described herein for high-boiling intermediate stream 58 are defined based on the total weight of organic components in the stream.

Stream 60 containing high molecular weight organics (oligomers) is drained from the middle boiler column 28 (e.g., a reboiler) and collected in the undesired high-boiling byproduct collection tank 62. From there the high molecular weight high boilers, stream 64, will be disposed of, possibly by sending them to a Thermal Oxidizer (T-OX) for incineration.

The hydrogen fluoride (HF) feed may be anhydrous. In other words, the hydrogen fluoride (HF) feed may be substantially free of water. The amount of water present in the hydrogen fluoride (HF) feed may be about 1000 ppm or less, about 750 ppm or less, about 500 ppm or less, about 250 ppm or less, about 100 ppm or less, about 50 ppm or less, about 25 ppm or less, about 10 ppm or less, about 5 ppm or less, or about 1 ppm or less, and for each of the foregoing, equal to or more than 0 ppm.

The temperature of the outlet of vaporizer/superheater 20 may be controlled to be as low as about 200 °C, about 220 °C, about 250 °C, about 300 °C, or as high as about 350 °C, about 400 °C, about 450 °C, about 500 °C, or within any range of the foregoing values as endpoints, for example, from about 200 °C to about 500 °C. Specific examples of additional suitable ranges are set forth below in Table 3. The values provided in Table 3 are understood to be prefaced by “about”.

TABLE 3
Temperature of Outlet of Vaporizer 20

| From (°C) | To (°C) |
|---|---|
| 200 | 500 |
| 200 | 450 |
| 200 | 400 |
| 200 | 350 |
| 220 | 500 |
| 220 | 450 |
| 220 | 400 |
| 220 | 350 |
| 250 | 500 |
| 250 | 450 |
| 250 | 400 |
| 250 | 350 |
| 300 | 500 |
| 300 | 450 |
| 300 | 400 |
| 300 | 350 |

The reaction in reactor 30 may be conducted in any suitable reaction vessel or reactor, but it should preferably be constructed from materials which are resistant to the corrosive effects of hydrogen fluoride such as nickel and its alloys, including Hastelloy, Inconel, Incoloy, and Monel. These may be single pipe or multiple tubes packed with a solid catalyst. Suitable reactors may include a catalytic pipe reactor, for example.

### Reaction Condition—Temperature

The temperature in reactor 30 may be as low as about 200 °C, about 210 °F, about 220 °C, about 230 °C, about 240 °C, or as high as about 250 °C, about 260 °C, about 270 °C, about 280 °C, about 290 °C, about 300 °C, about 350 °C, about 400 °C, or any value or range encompassed by these endpoints, for example, from about 200 °C to about 400 °C, or from about 210 °C to about 250 °C. Specific examples of additional suitable ranges are set forth below in Table 4. The values provided in Table 4 are understood to be prefaced by “about”.

TABLE 4
Reaction Temperature in Reactor 30

| From (°C) | To (°C) |
|---|---|
| 200 | 400 |
| 200 | 350 |
| 200 | 300 |
| 200 | 290 |
| 200 | 280 |
| 200 | 270 |
| 200 | 260 |
| 200 | 250 |
| 210 | 400 |
| 210 | 350 |
| 210 | 300 |
| 210 | 290 |
| 210 | 280 |
| 210 | 270 |
| 210 | 260 |
| 210 | 250 |
| 200 | 210 |
| 210 | 220 |
| 220 | 230 |
| 230 | 240 |
| 240 | 250 |
| 250 | 300 |
| 300 | 350 |
| 350 | 400 |

### Reaction Condition—Pressure

The pressure in reactor 30 may be maintained at about 2 psig or greater, about 5 psig or greater, about 10 psig or greater, about 15 psig or greater, about 20 psig or greater, about 25 psig or less, about 30 psig or less, about 35 psig or less, about 40 psig or less, about 45 psig or less, about 50 psig or less, about 75 psig or less, about 100 psig or less, or any value or range encompassed by these endpoints, for example, from about 2 psig to about 100 psig, or from about 5 psig to about 25 psig. Specific examples of additional suitable ranges are set forth below in Table 5. The values provided in Table 5 are understood to be prefaced by “about”.

TABLE 5
Reaction Pressure in Reactor 30

| From (psig) | To (psig) |
|---|---|
| 2 | 100 |
| 2 | 75 |
| 2 | 50 |
| 2 | 45 |
| 2 | 40 |
| 2 | 35 |
| 2 | 30 |
| 2 | 25 |
| 5 | 100 |
| 5 | 75 |
| 5 | 50 |
| 5 | 45 |
| 5 | 40 |
| 5 | 35 |
| 5 | 30 |
| 5 | 25 |
| 2 | 5 |
| 5 | 15 |
| 15 | 25 |
| 25 | 50 |
| 50 | 75 |
| 75 | 100 |

### Molar Ratio

The molar ratio of HF/(HCFC-241 isomers+HCFC-242 isomers) may be about 0.5/1 or higher, about 0.7/1 or higher, about 0.9/1 or higher, about 1.0/1 or higher, about 1.1/1 or higher, about 10.3/1 or higher, about 10.5/1 or higher, about 2.0/1 or less, about 2.5/1 or less, about 3.0/1 or less, about 3.5/1 or less, about 4.0/1 or less, about 5.0/1 or less, or any value or range encompassed by these endpoints, for example, from about 0.5/1 to about 5.0/1, or from about 0.7/1 to about 4.0/1.

### Catalysts

Suitable catalysts may include chromium oxides, chromium oxyfluorides, and chromium halides. The chromium oxides may include amorphous chromium oxide (Cr₂O₃), crystalline chromium oxide, and combinations of the foregoing. The chromium oxyfluorides may include fresh amorphous chromium oxide (Cr₂O₃) pretreated with HF, fresh crystalline chromium oxide (Cr₂O₃) pretreated with HF, amorphous chromium oxyfluoride (CrOₓF_{y}, where x may be greater than 0 but less than 1.5, and y may be greater than 0 but less than 3), crystalline chromium oxyfluoride (CrOₓF_{y}, where x may be greater than 0 but less than 1.5, and y may be greater than 0 but less than 3), and combinations of the foregoing. In one embodiment, the catalyst is amorphous chromium oxyfluoride (CrOₓF_{y}, where x may be greater than 0 but less than 1.5, and y may be greater than 0 but less than 3). The chromium halides may include chromium trifluoride (CrF₃), chromium trichloride (CrCl₃), chromium triiodide (Crl₃) and chromium tribromide (CrBr₃), and combinations of the foregoing. In one embodiment, the catalyst is chromium trifluoride (CrF₃).

Other suitable catalysts include promoted chromium-based catalysts, which are based on chromium and include an amount of at least one co-catalyst selected from Ni, Zn, Co, Mn, Mg, or mixtures thereof. The amount of the co-catalyst may be between 0.1 wt. % and 20 wt. % based on the total weight of the catalyst and, more particularly, may be present in an amount as little as 0.1 wt. %, 0.5 wt. %, 1.0 wt. %, 1.5 wt. %, or as high as 2.0 wt. %, 3.0 wt. %, 4.0 wt. %, 5.0 wt. %, 6.0 wt. %, or within any range using any two of the foregoing values as endpoints, based on to total weight of the catalyst. One suitable promoted chromium catalyst is a zinc/chromia catalyst which is based on chromia and includes an amount of zinc as a co-catalyst, for example, JM 62-3M catalyst available from Johnson Matthey. Prior to use, a fluorination treatment of the catalyst may be conducted using anhydrous HF under conditions effective to convert a portion of metal oxides into corresponding metal fluorides.

The above chromium-based catalysts may also be low chromium (VI) catalysts, having a total content of chromium (VI) oxide in an amount of about 50,000 ppm or less, about 40,000 ppm or less, about 30,000 ppm or less, about 20,000 ppm or less, about 10,000 ppm or less, about 5,000 ppm or less, about 2,000 ppm or less, about 1,000 ppm or less, about 500 ppm or less, about 250 ppm or less, or about 100 ppm or less, and for each of the foregoing, equal to or more than 0 ppm, based on total chromium oxides in the chromium oxide catalyst. Specific examples of additional suitable ranges are set forth below in Table 6. The values provided in Table 6 are understood to be prefaced by “about”.

TABLE 6
Chromium (VI) Oxide Content in Chromium-Based Catalysts

| From (ppm) | To (ppm) |
|---|---|
| ≥0 | 50,000 |
| ≥0 | 40,000 |
| ≥0 | 30,000 |
| ≥0 | 20,000 |
| ≥0 | 10,000 |
| ≥0 | 5,000 |
| ≥0 | 2,000 |
| ≥0 | 1,000 |
| ≥0 | 500 |
| ≥0 | 250 |
| ≥0 | 100 |

In addition to chromium-based catalysts, other suitable catalysts include alumina, iron oxide, magnesium oxide, zinc oxide, nickel oxide, cobalt oxide, aluminum fluoride or metal fluorides such as iron fluoride, magnesium fluoride, zinc fluoride, nickel fluoride, cobalt fluoride, fluorinated alumina, fluorinated iron oxide, fluorinated magnesium oxide, fluorinated nickel oxide, fluorinated cobalt oxide, titanium fluorides, molybdenum fluorides, aluminum oxyfluorides, and combinations of the foregoing. Prior to use, a fluorination treatment of catalyst containing metal oxide(s) is conducted using anhydrous HF under conditions effective to convert a portion of metal oxide(s) into corresponding metal fluoride(s).

### Product—Conversion

The collective percent conversion of 1,1,3,3-tetrachloro-1-fluoropropane (HCFC-241fa), HCFC-242 isomers (e.g., 1,3,3-trichloro-1,1-difluoropropane (HCFC-242fa), 1,1,3-trichloro-1,3-difluoropropane (HGFC-242fb)), HCFC-243 isomers (e.g., 3,3-dichloro-1,1,1-trifluoropropane (HCFC-243fa), and 1,3-dichloro-1,1,3-trifluoropropane (HCFC-243fb)) to 1-chloro-3,3,3-trifluoropropene (HCFO-1233zd) may be about 50% or greater, about 60% or greater, about 70% or greater, about 80% or greater, about 90% or greater, about 91% or greater, about 92% or greater, based on total amount of the product, and for each of the foregoing, less than or equal to 100%, or within any range using any two of the foregoing values as endpoints, for example, from about 50% to about 100%, or from about 60% to about 100%. Specific examples of additional suitable ranges are set forth below in Table 7. The values provided in Table 7 are understood to be prefaced by “about”.

TABLE 7
Conversion of 241fa, 242fa/fb, and 243fa/fb to 1233zdE

| From (%) | To (%) |
|---|---|
| 50 | ≤100 |
| 60 | ≤100 |
| 70 | ≤100 |
| 75 | ≤100 |
| 80 | ≤100 |
| 90 | ≤100 |
| 91 | ≤100 |
| 92 | ≤100 |
| 50 | 60 |
| 60 | 70 |
| 70 | 80 |
| 80 | 90 |
| 90 | 91 |
| 91 | 92 |
| 92 | 93 |

### Product—Selectivity

The selectivity for E-1-chloro-3,3,3-trifluoropropene (HCFO-1233zdE) may be about 60% or greater, about 65% or greater, about 70% or greater, about 72% or greater, about 74% or greater, about 76% or greater, about 78% or greater, based on total amount of the product, and for each of the foregoing, less than or equal to 100%, or within any range using any two of the foregoing values as endpoints, for example, from about 70% to about 100%, or from about 72% to about 100%. Specific examples of additional suitable ranges are set forth below in Table 8. The values provided in Table 8 are understood to be prefaced by “about”.

TABLE 8
Selectivity for 1233zdE

| From (%) | To (%) |
|---|---|
| 60 | ≤100 |
| 65 | ≤100 |
| 70 | ≤100 |
| 72 | ≤100 |
| 74 | ≤100 |
| 76 | ≤100 |
| 78 | ≤100 |
| 70 | 72 |
| 72 | 74 |
| 74 | 76 |
| 76 | 78 |
| 78 | 80 |

While E-1-chloro-3,3,3-trifluoropropene HCFO-1233zd(E)) may be separated and purified as a product, any Z-1-chloro-3,3,3-trifluoropropene HCFO-1233zd(Z)) and 3-chloro-1,1,1-3-tetrafluoropropane (HCFC-244fa) produced may be separated/recovered and fed to a combination isomerization/dehydrofluorination reactor to be further converted to E-1-chloro-3,3,3-trifluoropropene HCFO-1233zd(E)).

### EXAMPLES

### Example 1

### Conversion of 1233zdE High Boilers and HF to 1233zdE

This example includes the reactor effluent organic analysis of the 1233zdE crude product produced from the reaction of 1233zdE high boilers and HF to produce 1233zdE. The process flow is described above with regard to FIG. 1. The reactions that occur in the reactor 30 may include those according to Equations 6-14. The reactor used for Example 1 was a tubular Monel reactor (ID=2″). The reactor was charged with 0.7 L JM-62 chromium oxyfluoride catalyst and placed inside a sand bath.

Anhydrous hydrogen fluoride (AHF) and 1233zdE high boilers were vaporized and then fed continuously to the reactor. The reaction conditions were as follows: HF feed rate was 25 g/hr, 1233zdE high boiler feed rate was 260 g/hr, the AHF to 1233zdE high boiler ratio was 0.90:1, contact time was 40 s, catalyst hot spot temperature was 221 °C, and pressure within the reactor was 10 psig.

The 1233xf selectivity or concentration in the reactor effluent was consistently analyzed to be 0.048 mole % or 0.0426 GC area %. The catalyst productivity was 9.5 lb/hr/ft³ of catalyst. The collective conversion of 1233zdE high boiler components 241fa, 242fa/fb, and 243fa was about 92.0 mole % and the average 1233zdE selectivity was 70.7 mole %. The average selectivities for 1234ze(E), 245fa, 244fa were 0.6 mole %, 1.61 mole %, and 2.53 mole %, respectively. The average selectivity to 1233zd(Z) was 9.2 mole %. The total raw amount of recyclable compounds which include 243fb, 243fa, 242fa/fb, 241fa,1232zd isomers, and 1231 isomers (assumed to be zd isomers) averaged 14.73 GC area %. The reaction data is shown below in Table 12, and the GC analysis of the 1233zdE high boiler feed is shown in Table 9.

TABLE 9
1233zdE high boiler feed stock GC data

| | | Retention Time | | |
|---|---|---|---|---|
| Peak# | Component | (Min) | Area | Area % |
| 1 | FC-1233zdE | 13.985 | 52391.4 | 0.0437 |
| 2 | FC-244fa | 15.159 | 387835. | 0.3931 |
| 3 | FC-1233zdZ | 16.212 | 939517 | 1.5065 |
| 4 | FC-1232 (1) | 18.013 | 24476.0 | 0.0565 |
| 5 | FC-1232 (2) | 18.281 | 90920.9 | 0.0529 |
| 6 | FC-243 (1) | 18.476 | 2903280 | 0.7017 |
| 7 | FC-243 (2) | 18.653 | 2553248 | 5.7105 |
| 8 | FC-1232 (3) | 19.368 | 11133.0 | 0.0388 |
| 9 | FC-1232 (4) | 19.423 | 9075.25 | 0.0173 |
| 10 | FC-1233 | 19.505 | 488143. | 0.3014 |
| 11 | FC-242fa | 21.174 | 3221842 | 59.2322 |
| 12 | FC-242fb | 21.267 | 2272552 | 6.0527 |
| 13 | FC-1231 (1) | 21.317 | 61265.1 | 0.1167 |
| 14 | FC-1231 (2) | 21.71 | 4799.55 | 0.0127 |
| 15 | FC-241fa | 23.279 | 9616269 | 25.3132 |
| 16 | FC-240fa | 24.917 | 6778.63 | 0.0182 |

| | |
|---|---|
| Unidentified peaks total | 0.3322 |

### Example 2

### Purification of the 1233zdE Crude Product Produced from the Reaction of 1233zdE Hiqh Boilers and HF

This example includes the purification of the 1233zdE crude product produced from the reaction of 1233zdE high boilers and HF to produce 1233zdE encompassing the removal of low boiling and high boiling co-products.

After collecting the reactor effluent from Example 1 into a batch distillation column for about 260 hours, a batch distillation was conducted on the material. HCl was continuously vented to keep a distillation column pressure of between 8 and 10 psig while the reaction was running. The batch distillation column had a 10 gallon reboiler attached to a 2″ ID x 10′ L distillation column with a condenser. The column was packed with random packing and had about 30 theoretical stages. The batch column was equipped with temperature, pressure, and differential pressure instruments.

There were multiple distillation cuts taken overhead to simulate the 2 continuous distillation columns (e.g., distillation column 50 and middle boiler column 28 as shown in FIG. 1) of the present disclosure. The first cut was the removal of the remaining HCl, unreacted HF and the low boiling organic components including 1233zdE, 245fa, 1234zeE, 1234zeZ, and 1233xf which were passed through a scrubber to remove the acid, dried, and collected. These were the components that would be removed overhead off the first continuous distillation column (e.g., distillation column 50 as shown in FIG. 1). About 95 lbs of crude 1233zdE was collected.

The second cut removed most of the 244fa and 1233zdZ which have similar normal boiling points. These would also be removed in the first continuous distillation column (e.g., distillation column 50 as shown in FIG. 1).

A third cut removed 241fa, 242fa, 242fb, 243fa, 243fb, 1232 and 1231 isomers overhead and left behind about 4.5 lbs of high molecular weight high boilers in the reboiler which were drained and disposed of. The third cut would be taken off in the second continuous distillation column (e.g., middle boiler column 28 as shown in FIG. 1). The third cut amounted to 30 lbs of recyclable 1233zdE high boilers were collected in the third cut. The GC analysis of the third cut can be found in Table 10.

TABLE 10
GC analysis of third cut of batch distillation

| | | Retention | | |
|---|---|---|---|---|
| Peak# | Component | Time | Area | Area % |
| 1 | FC-244fa | 15.16 | 664612.1 | 0.5902 |
| 2 | FC-1233zdZ | 16.2 | 368228.1 | 0.327 |
| 3 | FC-1232 (1) | 18.034 | 24347.64 | 0.0216 |
| 4 | FC-1232 (2) | 18.314 | 2001090.8 | 1.777 |
| 5 | FC-243fa | 18.646 | 25506136. | 23.4494 |
| 6 | FC-243fb | 18.774 | 6402177 | 5.8851 |
| 7 | FC-1232 (3) | 19.396 | 39481.69 | 0.0351 |
| 8 | FC-1232 (4) | 19.463 | 133222.9 | 0.1183 |
| 9 | FC-1233 dimer | 19.566 | 2818456.4 | 2.5028 |
| 10 | FC-242fa | 21.277 | 69706468. | 63.8993 |
| 11 | FC-242fb | 21.377 | 286414.34 | 0.2543 |
| 12 | FC-1231 (1) | 21.525 | 172888.19 | 0.1535 |
| 13 | FC-1231 (2) | 21.668 | 39489.72 | 0.0351 |
| 14 | FC-241fa | 23.187 | 497305.69 | 0.5416 |

| | |
|---|---|
| Unidentified High Boiler peaks total | 0.4097 |

### Example 3

### Conversion of 1233zdE High Boilers and Recyclable 1233zdE High Boilers and HF to 1233zdE

The process in Example 1 was repeated with a different feed stock. This example includes the reactor effluent organic analysis of the 1233zdE crude product produced from the reaction of 1233zdE high boilers and HF to produce 1233zdE. This was mixed with 96 lbs of 1233zdE high boilers to make up a feed stock having 76.2% 1233zdE high boilers (same composition as the feedstock in Example 1) and 23.8% recyclable 1233zdE high boilers (same composition as the recyclable 1233zdE high boilers collected in the third cut in Example 2).

Anhydrous hydrogen fluoride (AHF) and the mixture of 1233zdE high boilers and recyclable 1233zdE high boilers were vaporized and then fed continuously to the reactor.

The reaction using this feed cylinder was run continuously at the same reaction conditions as used in Example 1. The reaction conditions were as follows: HF feed rate was 25 g/hr, 1233zdE high boiler feed rate was 260 g/hr, the AHF to 1233zdE high boiler ratio was 0.90:1, contact time was 40 s, catalyst hot spot temperature was 221 °C, and pressure within the reactor was 10 psig. 0.7 L catalysts were used. The reaction was run continuously for 125 hours. The average 1233xf formation was 0.0306 GC area %, equivalent to 0.037 mole %. This was significantly lower than the result in Example 1 using the 100% 1233zdE high boiler feed stock. GC analysis of the mixed feed stock used in Example 3 is shown in Table 11.

TABLE 11
GC analysis of the mixture of 1233zdE high boilers (76.2
wt %) and recyclable 1233zdE High Boilers (23.8 wt %)

| | | Retention | | |
|---|---|---|---|---|
| | | Time | | |
| Peak# | Component | (Min) | Area | Area % |
| 1 | FC-1233zd | 14.015 | 1163.3 | 0.0114 |
| 2 | FC-244fa | 15.133 | 59921.8 | 0.5892 |
| 3 | FC-1233zd Cis | 16.176 | 102643.2 | 1.0094 |
| 4 | FC-1232 (1) | 17.998 | 4799.55 | 0.0472 |
| 5 | FC-1232 (2) | 18.263 | 56239.27 | 0.553 |
| 6 | FC-243 (1) | 18.421 | 705452.5 | 6.9372 |
| 7 | FC-243 (2) | 18.593 | 722945.9 | 7.5092 |
| 8 | FC-1232 (3) | 19.398 | 2952.09 | 0.029 |
| 9 | FC-1232 (4) | 19.438 | 5695.45 | 0.056 |
| 10 | FC-1233 dimer | 19.473 | 93201.66 | 0.9165 |
| 11 | FC-242fa | 21.021 | 6292629. | 61.4795 |
| 12 | FC-242fb | 21.131 | 451753.7 | 4.4424 |
| 13 | FC-1231 (1) | 21.261 | 10950.2 | 0.1077 |
| 14 | FC-1231 (2) | 21.515 | 2993.91 | 0.0294 |
| 15 | FC-241fa | 23.172 | 1493787. | 14.7641 |
| 16 | FC-240fa | 24.917 | 6778.63 | 0.0701 |

| | |
|---|---|
| Unidentified peaks total | 1.3786 |

The collective conversion of the major 1233zd high boiler feed components, 241fa, 242fa/fb, and 243fa was about 89.6 mole % and the average 1233zd(E) selectivity was 78.8 mole %. The average selectivities for 1234ze(E), 245fa, 244fa were 0.69 mole %, 2.1 mole %, and 3.6 mole %, respectively. The average selectivity to 1233zd(Z) was 10.0 mole % which was equal to the result in Example 1 using the 100% 1233zdE high boiler feed stock. The total raw amount of recyclable compounds which include 243fb, 243fa, 242fa/fb, 241fa,1232zd isomers, and 1231 isomers averaged 18.3 GC area % which is higher than those in Example 1.

The comparison between the reaction data for Example 1 and Example 3 are shown below in Table 12.

TABLE 12
Summary of Co-product selectivity and 1233zdE High
Boiler conversion data of reactor effluent samples.

| | | Recycle - |
|---|---|---|
| | | 75/25% 1233zdE |
| | 1233zd High | High Boilers/Recycled |
| | Boilers neat | 1233zdE |
| Feed Stock | feed | High Boilers |
| 1234zeE | 0.55 | 0.69 |
| sel (mol %) | | |
| 245fa | 1.61 | 2.10 |
| sel (mol %) | | |
| 1234zeZ | 0.10 | 0.14 |
| sel (mol %) | | |
| 1233xf | 0.048 | 0.037 |
| sel (mol %) | | |
| 1233zdE | 79.44 | 78.78 |
| sel (mol %) | | |
| 244fa | 2.53 | 3.59 |
| sel (mol %) | | |
| 1233zdZ | 9.19 | 9.96 |
| sel (mol %) | | |
| 1232 iso(s) | 2.03 | 2.32 |
| sel (mol %) | | |
| 243fb | 4.30 | 2.10 |
| sel (mol %) | | |
| 1233 dimer | 0.00 | 0.00 |
| sel (mol %) | | |
| 1231 iso(s) | 0.04 | 0.04 |
| sel (mol %) | | |
| others | 0.24 | 0.25 |
| sel (mol %) | | |
| 243fa | 70.68 | 66.85 |
| conversion | | |
| (mol %) | | |
| 242fa | 90.90 | 88.87 |
| conversion | | |
| (mol %) | | |
| 241fa | 99.82 | 99.50 |
| conversion | | |
| (mol %) | | |
| 240fa | 100.0 | 100.0 |
| conversion | | |
| (mol %) | | |
| Overall | 92.04 | 89.08 |
| conversion | | |
| (mol %) | | |
| ¹Recyclables | 14.73 | 18.31 |
| left GC area % | | |

### Example 4

### Per Se Composition of High-boiling Intermediate Stream

This example describes a composition of a high-boiling intermediate stream from a middle boiler column which may be fed back into a feed tank. The high-boiling intermediate stream comprises primarily 241fa, 242fa, 242fb, 243fa, 243fb, 1232 and 1231 isomers.

The 1233zdE product is purified over multiple trials according to Example 2, and a sample of the third cut of the batch distillation (high-boiling intermediate stream) is taken and analyzed to determine its components. Over the course of the multiple trials, components are present in the ranges presented below in Table 13.

TABLE 13
Summary of Product Composition of
High-boiling Intermediate Stream

| Component | From (wt. %) | To (wt. %) |
|---|---|---|
| HCFC-242fa | 10 | 90 |
| HCFC-243fa | 5 | 50 |
| HCFC-243b | 0.5 | 15 |
| HCFC-1233 | 0.1 | 10 |
| HCFC-1232 | 0.1 | 10 |
| Other components | 0.1 | 10 |

### Aspects

Aspect 1 is a method for producing E-1-chloro-3,3,3-trifluoropropene (HCFO-1233zd(E)), comprising: providing a mixture of high-boiling fluorinated components having a normal boiling point of greater than 39 °C; contacting the mixture of high-boiling fluorinated components with hydrogen fluoride (HF) to produce a combined reactant stream; reacting the combined reactant stream in the presence of a catalyst to produce a product stream comprising E-1-chloro-3,3,3-trifluoropropene (HCFO-1233zd(E)); recovering, from the product stream, a high-boiling intermediate stream comprising recyclable high-boiling intermediates having a normal boiling point of greater than 39 °C and less than 140 °C; and recycling the recyclable high-boiling intermediates into the mixture of high-boiling fluorinated components prior to the reacting step.

Aspect 2 is the method of Aspect 1, further comprising, after the contacting step and before the reacting step: vaporizing the combined reactant stream to produce a vaporized reactant stream and high-boiling byproducts with a normal boiling point of greater than 140 °C; and separating the high-boiling byproducts from the vaporized reactant stream.

Aspect 3 is the method of Aspects 1 or 2, wherein the recovering step comprises removing at least a portion of high-boiling byproducts with a normal boiling point of greater than 140 °C produced in the reacting step from the product stream to produce a first processed stream.

Aspect 4 is the method of Aspect 3, wherein the removing step comprises condensing, and purging from the product stream, at least a portion of the high-boiling byproducts with a normal boiling point of greater than 140 °C.

Aspect 5 is the method of Aspect 4, wherein the condensing step is conducted at a temperature of 10 °C or higher.

Aspect 6 is the method of any one of Aspects 3-5, wherein the recovering step further comprises separating a second product stream comprising HCFO-1233zd(E) from the first processed stream to produce a second processed stream.

Aspect 7 is the method of Aspect 6, wherein the second product stream further comprises low boiling impurities with a normal boiling point of less than 39 °C, HCl, and unreacted HF.

Aspect 8 is the method of Aspect 7, wherein the recovering step further comprises removing the low boiling impurities, HCl, and unreacted HF from the second product stream to produce a purified product stream.

Aspect 9 is the method of Aspects 7 or 8, wherein the low boiling impurities are selected from 1,1,1,3,3-pentafluoropropane (HFC-245fa), trifluoropropyne, E- or Z-isomers of 1,3,3,3-tetrafluoropropene (HFO-1234ze), 2-chloro-3,3,3-trifluoropropene (HCFO-1233xf), and combinations thereof.

Aspect 10 is the method of any one of Aspects 6-9, wherein the recovering step further comprises removing 3-chloro-1,1,1,3-tetrafluoropropane (HCFC-244fa), Z-1-chloro-3,3,3-trifluoropropene (HCFO-1233zd(Z)) from the second processed stream to produce a third processed stream.

Aspect 11 is the method of Aspect 10, wherein the recovering step further comprises removing high-boiling byproducts with a normal boiling point of greater than 140 °C from the third processed stream to produce the high-boiling intermediate stream comprising recyclable high-boiling intermediates.

Aspect 12 is the method of any one of Aspects 2-11, wherein the high-boiling byproducts are selected from oligomers, hexachloroethane, and combinations thereof.

Aspect 13 is the method of any one of Aspects 3-12, wherein the one or more removing steps comprise distilling the product stream.

Aspect 14 is the method of any one of Aspects 1-13, wherein the high-boiling intermediate stream comprises one or more recyclable high-boiling intermediates selected from 1,1,3,3-tetrachloro-1-fluoropropane (HCFC-241fa), 1,3,3-trichloro-1,1-difluoropropane (HCFC-242fa), and 3,3-dichloro-1,1,1-trifluoropropane (HCFC-243fa), isomers thereof, and combinations thereof.

Aspect 15 is the method of any one of Aspects 1-14, wherein the mixture of high-boiling fluorinated components comprises 1,1,3,3-tetrachloro-1-fluoropropane (HCFC-241fa), 1,3,3-trichloro-1,1-difluoropropane (HCFC-242fa), 1,1,3-trichloro-1,3-difluoropropane (HCFC-242fb), 3,3-dichloro-1,1,1-trifluoropropane (HCFC-243fa), 1,3-dichloro-1,1,3-trifluoropropane (HCFC-243fb), 3-chloro-1,1,1,3-tetrafluoropropane (HCFC-244fa), and combinations thereof.

Aspect 16 is the method of any one of Aspects 2-15, wherein the vaporizing step is conducted at a temperature of 200 °C or higher.

Aspect 17 is the method of any one of Aspects 1-16, wherein the reacting step is conducted at a temperature between 200 °C and 300 °C.

Aspect 18 is the method of any one of Aspects 1-17, wherein the mixture of high boiling fluorinated components comprises components derived from reacting HCC-240fa to form HFO-1233zd.

Aspect 19 is the method of any one of Aspects 1-18, wherein the catalyst is selected from the group consisting of chromium oxides, chromium oxyfluorides, and chromium halides.

Aspect 20 is the method of any one of Aspects 1-19, wherein the catalyst is a chromium oxyfluoride catalyst.

Aspect 21 is the method of any one of Aspects 1-20, wherein the catalyst is a zinc/chromium catalyst including 0.5 to 6.0 wt. % Zn, based on the total weight of the catalyst.

Aspect 22 is the method of any one of Aspects 1-21, further comprising at least one of the following additional steps: combining the product stream with a crude stream comprising E-1-chloro-3,3,3-trifluoropropene (HCFO-1233zd(E)) to produce a combined product stream; and distilling the combined product stream to obtain a recyclable stream comprising recyclable intermediates; wherein the recyclable intermediates comprise at least one selected from the group consisting of 1,1,3,3-tetrachloro-1-fluoropropane (HCFC-241fa), 1,3,3-trichloro-1,1-difluoropropane (HCFC-242fa), 1,1,3-trichloro-1,3-difluoropropane (HCFC-242fb), 3,3-dichloro-1,1,1-trifluoropropane (HCFC-243fa), 1,3-dichloro-1,1,3-trifluoropropane (HCFC-243fb), and 3-chloro-1,1,1,3-tetrafluoropropane (HCFC-244fa), 1,1,3,3-tetrachloropropene (HCO-1230za), and their isomers.

Aspect 23 is the method of any one of Aspects 2-22, wherein: the vaporizing step is conducted at a temperature of 200 °C or higher; the condensing step is conducted at a temperature of 10 °C or higher; and the catalyst is chromium oxide.

Aspect 24 is the method of any one of Aspects 1-23, wherein the catalyst is a zinc/chromium catalyst including 0.5 to 6.0 wt. % Zn, based on the total weight of the catalyst.

Aspect 25 is the method of any one of Aspects 1-24, wherein the high-boiling byproducts comprise at least one of hexachloroethane (CC-110), tetrachloropropene (HCO-1230) isomers, dimers of 1,3,3-trichloro-3-fluoropropene, dimers of 1,2,-dichloro-3,3-difluoropropene, and combinations thereof.

Aspect 26 is the method of any one of Aspects 1-25, wherein the hydrogen fluoride includes 1000 ppm or less of water.

Aspect 27 is the method of any one of Aspects 2-26, wherein the vaporized reactant stream comprises a molar ratio of hydrogen fluoride (HF) to HCFC-241 and HCFC-242 isomers of from 0.9:1 to 5.0:1.

Aspect 28 is the method of any one of Aspects 1-27, wherein the catalyst comprises chromium (VI) oxide in an amount of 5000 ppm or less, based on a total amount of chromium oxides in the catalyst.

Aspect 29 is the method of any one of Aspects 1-28, wherein the high-boiling byproducts intermediates comprise: HCFC-242 fa from about 10 wt. % to about 90 wt. %; HCFC-243fa from about 5 wt. % to about 50 wt. %; HCFC-243b from about 0.5 wt. % to about 15 wt. %; HCFC-1233 from about 0.1 wt. % to about 10 wt. %; and HCFC-1232 (2) from about 0.1 wt. % to about 10 wt. %.

Aspect 30 is A composition comprising E-1-chloro-3,3,3-trifluoropropene (HCFO-1233zd(E)) made according to the method of any one of Aspects 1-29. While this disclosure has been described as relative to exemplary designs, the present disclosure may be further modified within the spirit and scope of this disclosure. Further, this application is intended to cover such departures from the present disclosure as come within known or customary practice in the art to which this disclosure pertains.

1. A method for producing E-1-chloro-3,3,3-trifluoropropene (HCFO-1233zd(E)), comprising:
providing a mixture of high-boiling fluorinated components having a normal boiling point of greater than 39 °C;
contacting the mixture of high-boiling fluorinated components with hydrogen fluoride (HF) to produce a combined reactant stream;
reacting the combined reactant stream in the presence of a catalyst to produce a product stream comprising E-1-chloro-3,3,3-trifluoropropene (HCFO-1233zd(E));
recovering, from the product stream, a high-boiling intermediate stream comprising recyclable high-boiling intermediates having a normal boiling point of greater than 39 °C and less than 140 °C; and
recycling the recyclable high-boiling intermediates into the mixture of high-boiling fluorinated components prior to the reacting step.

2. The method of claim 1, further comprising, after the contacting step and before the reacting step:
vaporizing the combined reactant stream to produce a vaporized reactant stream and high-boiling byproducts with a normal boiling point of greater than 140 °C; and
separating the high-boiling byproducts from the vaporized reactant stream.

3. The method of claim 1, wherein the recovering step comprises:
removing at least a portion of high-boiling byproducts with a normal boiling point of greater than 140 °C produced in the reacting step from the product stream to produce a first processed stream;
condensing at a temperature of 10 °C or higher; and
purging from the product stream, at least a portion of the high-boiling byproducts with a normal boiling point of greater than 140 °C.

4. The method of claim 3, wherein the recovering step further comprises separating a second product stream comprising HCFO-1233zd(E) from the first processed stream to produce a second processed stream.

5. The method of claim 4, wherein the second product stream further comprises low boiling impurities with a normal boiling point of less than 39 °C, HCl, and unreacted HF.

6. The method of claim 5, wherein the recovering step further comprises removing the low boiling impurities, HCl, and unreacted HF from the second product stream to produce a purified product stream.

7. The method of claim 6, wherein the low boiling impurities are selected from 1,1,1,3,3-pentafluoropropane (HFC-245fa), trifluoropropyne, E- or Z-isomers of 1,3,3,3-tetrafluoropropene (HFO-1234ze), 2-chloro-3,3,3-trifluoropropene (HCFO-1233xf), and combinations thereof.

8. The method of claim 4, wherein the recovering step further comprises:
removing 3-chloro-1,1,1,3-tetrafluoropropane (HCFC-244fa), Z-1-chloro-3,3,3-trifluoropropene (HCFO-1233zd(Z)) from the second processed stream to produce a third processed stream; and
removing high-boiling byproducts with a normal boiling point of greater than 140 °C from the third processed stream to produce the high-boiling intermediate stream comprising recyclable high-boiling intermediates.

9. The method of claim 2, wherein the high-boiling byproducts are selected from oligomers, hexachloroethane, and combinations thereof.

10. The method of claim 3, wherein the one or more removing steps comprise distilling the product stream.

11. The method of claim 1, wherein the high-boiling intermediate stream comprises one or more recyclable high-boiling intermediates selected from 1,1,3,3-tetrachloro-1-fluoropropane (HCFC-241fa), 1,3,3-trichloro-1,1-difluoropropane (HCFC-242fa), and 3,3-dichloro-1,1,1-trifluoropropane (HCFC-243fa), isomers thereof, and combinations thereof.

12. The method of claim 1, wherein the mixture of high-boiling fluorinated components comprises 1,1,3,3-tetrachloro-1-fluoropropane (HCFC-241fa), 1,3,3-trichloro-1,1-difluoropropane (HCFC-242fa), 1,1,3-trichloro-1,3-difluoropropane (HCFC-242fb), 3,3-dichloro-1,1,1-trifluoropropane (HCFC-243fa), 1,3-dichloro-1,1,3-trifluoropropane (HCFC-243fb), 3-chloro-1,1,1,3-tetrafluoropropane (HCFC-244fa), and combinations thereof.

13. The method of claim 2, wherein the vaporizing step is conducted at a temperature of 200 °C or higher and the reacting step is conducted at a temperature between 200 °C and 300 °C.

14. The method of claim 1, wherein the mixture of high boiling fluorinated components comprises components derived from reacting HCC-240fa to form HFO-1233zd.

15. The method of claim 1, wherein the catalyst is selected from the group consisting of chromium oxides, chromium oxyfluorides, and chromium halides.

16. The method of claim 1, further comprising at least one of the following additional steps:
combining the product stream with a crude stream comprising E-1-chloro-3,3,3-trifluoropropene (HCFO-1233zd(E)) to produce a combined product stream; and
distilling the combined product stream to obtain a recyclable stream comprising recyclable intermediates;
wherein the recyclable intermediates comprise at least one selected from the group consisting of 1,1,3,3-tetrachloro-1-fluoropropane (HCFC-241fa), 1,3,3-trichloro-1,1-difluoropropane (HCFC-242fa), 1,1,3-trichloro-1,3-difluoropropane (HCFC-242fb), 3,3-dichloro-1,1,1-trifluoropropane (HCFC-243fa), 1,3-dichloro-1,1,3-trifluoropropane (HCFC-243fb), and 3-chloro-1,1,1,3-tetrafluoropropane (HCFC-244fa), 1,1,3,3-tetrachloropropene (HCO-1230za), and their isomers.

17. The method of claim 2, wherein:
the vaporizing step is conducted at a temperature of 200 °C or higher;
the condensing step is conducted at a temperature of 10 °C or higher; and
the catalyst is chromium oxide.

18. The method of claim 1, wherein the high-boiling intermediates comprise:
HCFC-242 fa from about 10 wt. % to about 90 wt. %;
HCFC-243fa from about 5 wt. % to about 50 wt. %;
HCFC-243b from about 0.5 wt. % to about 15 wt. %;
HCFC-1233 from about 0.1 wt. % to about 10 wt. %; and
HCFC-1232 (2) from about 0.1 wt. % to about 10 wt. %.

19. The method of claim 2, wherein the vaporized reactant stream comprises a molar ratio of hydrogen fluoride (HF) to HCFC-241 and HCFC-242 isomers of from 0.9:1 to 5.0:1.

20. A composition comprising E-1-chloro-3,3,3-trifluoropropene (HCFO-1233zd(E)) made according to the method of claim 1.
